(19) United States
(12) Patent Application Publication
(10) Pub. No.: US 2025/0283965 A1
(43) Pub. Date: Sep. 11, 2025

(54) COMPUTER-IMPLEMENTED METHOD FOR OPERATING A MAGNETIC RESONANCE DEVICE, MAGNETIC RESONANCE DEVICE, COMPUTER PROGRAM AND ELECTRONICALLY READABLE DATA MEDIUM

(71) Applicant: Siemens Healthineers AG, Forchheim (DE)

(72) Inventors: Mario Zeller, Erlangen (DE); Peter Speier, Erlangen (DE)

(21) Appl. No.: 19/074,380

(22) Filed: Mar. 8, 2025

(30) Foreign Application Priority Data
Mar. 8, 2024 (DE) 10 2024 202 215.2

(51) Int. Cl. G01R 33/56; G06T 11/00; G16H 40/60

(52) U.S. Cl. CPC G01R 33/5608; G06T 11/005; G16H 40/60

(57) ABSTRACT

In a computer-implemented method, at least one measurement value that describes a reflected power reflected back by a coil element that is used to output radiofrequency pulses is acquired for at least some of the radiofrequency pulses in each case in a measurement interval such that a time series of measurement values is produced over the acquisition period. The time series of measurement values is evaluated for the purpose of detecting an occurring movement of a scanned examination subject. The movement is described by motion information. The motion information is used for controlling the acquisition procedure and/or in a reconstruction of an image dataset from acquired magnetic resonance data. The time series is used prior to the evaluation for at least partially cleaning the time series of the property using at least one piece of background information that describes an expected, non-motion-related property of the time series.

This application claims the benefit of German Patent Application No. DE 10 2024 202 215.2, filed on Mar. 8, 2024, which is hereby incorporated by reference in its entirety.

### BACKGROUND

The present embodiments relate to operating a magnetic resonance device during an acquisition procedure in accordance with an acquisition protocol.

Magnetic resonance imaging has become a routinely employed diagnostic and monitoring tool in medical application. Due to the relatively long acquisition period of conventional acquisition protocols for magnetic resonance data, motion is an important topic with regard to improving image quality since movements during the acquisition time, even in the event of correction, may lead to losses in image quality and consequently also in the usability of the magnetic resonance data as well as of image datasets reconstructed therefrom. In this regard, when a patient is scanned, motion relates both to cyclical motion processes in the living examination subject and to other, wanted and unwanted, externally and internally triggered motion processes of the acquisition region.

The avoidance or consideration of motion in Turbo Spin Echo sequences (TSE sequences; also known as HASTE sequences or FSE sequences) is proving to be particularly relevant. In a TSE sequence, a radiofrequency excitation pulse is followed by a pulse sequence known as an echo train in which respective refocusing radiofrequency pulses are followed in each case by readout time intervals, all of which benefit from the same excitation radiofrequency pulse. This results in acquisition periods of a number of minutes in the case of multiple echo trains. Even so, the TSE sequence is still employed, owing to its merits as a “workhorse” of medical imaging.

In an article by D. Buikman et al., “The RF coil as a sensitive motion detector for magnetic resonance imaging,” Magnetic Resonance Imaging 6 (1988), pages 218-289, it is proposed to evaluate the reflected power reflected back by coil elements of a radiofrequency coil array that is used for transmitting radiofrequency pulses in order to detect movements of a patient. Cyclical movements (e.g., respiratory motions and cardiac motion) are detected in this case. Non-periodic movements (e.g., swallowing or coughing) may also be detected, which provides that magnetic resonance data degraded due to motion may be discarded, then enabling fresh data to be acquired again during motionless time periods.

However, the approach described there allows only a rough assessment, which, for example, permits data affected by strong movement, as occurs with the heartbeat for example, to be discarded or a triggering. A more accurate evaluation (e.g., with regard to non-periodic and/or weaker movements) is not possible due to further influencing factors, which may also lead to differences in measurement values.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, improved (e.g., more reliable, more robust, and higher-quality) detection of movements using a radiofrequency coil array is provided.

In a method, it is provided according to the present embodiments that the time series, using at least one piece of background information describing an expected property of the time series that is not related to movement, is used in a preprocessing step prior to the evaluation step for at least partially cleaning the time series of the property.

The present embodiments, too, therefore use the acquisition of a time series of measurement values of the reflected power reflected back by used coil elements of the radiofrequency coil array, where, for example, a directional coupler may be used for the measurement. If the coil element is also used anyway for receiving magnetic resonance signals, the receive electronics may also be activated during the transmission of the radiofrequency pulse for which a measurement value is to be acquired. Otherwise, a dedicated measurement electronics device may be provided. Since measurements of this type are already known in other application fields (e.g., for long-term stabilization of the radiofrequency pulse voltage in demanding diffusion sequences), it is a general advantage that no modification is necessary on the magnetic resonance device (e.g., no additional hardware or even any modification of the timing sequence in the acquisition protocol, such as of the sequence timing). Thus, the use of the method described, for example, does not lead to a prolonging of the acquisition period or to changes to the workflow of the acquisition program since only the radiofrequency pulses are to be provided with a marker, and the acquisition is controlled by the control device (e.g., by the framework).

In this case, measurement values may, for example, be acquired during a longer portion of or indeed of the entire time interval in which the radiofrequency pulse is output. This provides that the measurement interval then corresponds to the duration of the output of the radiofrequency pulse. In practice, it may be provided that a number of measurements are performed in each measurement interval, a measurement value representative of the measurement interval being determined by statistical consolidation (e.g., averaging) of the measurement results. Multiple representative measurement values may also be determined in this way. In one embodiment, each of the measurement values may be retained. Since an analog-to-digital converter (ADC) of the measurement electronics device is typically switched to “open” for the measurement in magnetic resonance devices, the measurement interval may also be referred to as “TX-ADC” (in contrast to an “RX-ADC” for the acquisition time interval).

In one embodiment, prior knowledge may be incorporated in addition to background information, which improves the evaluations of the time series of measurement values for detecting motion. Prior to the evaluation step, a preprocessing step is performed in which the background information is used in order already to remove, at least to some extent, differences in the measurement values that are not due to movements. In other words, the background information therefore describes an expected change in the measurement values over time in the time series as an expected property of the time series. The identifiability of motion may be greatly improved by the incorporation of this prior knowledge.

In this case, the method may be employed in many types of magnetic resonance sequences (e.g., when the magnetic resonance sequence is a TSE sequence in which individual acquisition time intervals may be identified as affected by movement such as within echo train)s. However, the fact that individual acquisition time intervals, and consequently also individual k-space trajectory sections (e.g., k-space lines), may be identified as affected by movement also permits the applicability of the method in other sequence types (e.g., in gradient echo sequences (GRE sequences), multi-echo sequences, and the like).

The measurement values may be regarded as B1 reflection raw data that may be corrected in the preprocessing step using prior knowledge about the expected profile. The proposed approach permits a plethora of potential applications, which will be explored in greater depth in the following. The motion information may be determined reliably, robustly, and with high quality. When a distinction is made below between “movement” and “no movement”, this is understood in terms of the relevance of the movement. Relevant movement may be present, for example, when a relevance condition for movement described by the motion information (e.g., its strength) is met.

Radiofrequency pulses for which measurement values of the back-reflected reflected power are to be acquired may be marked in the acquisition protocol. A sequence unit of a control device of the magnetic resonance device that controls the acquisition operation may also control the acquisition of the measurement values for correspondingly marked radiofrequency pulses. In this case, a beneficial development of the present embodiments provides that at least one measurement value is acquired at least for each radiofrequency pulse (e.g., immediately) preceding an acquisition time interval. Thus, there are measurement values available for all acquisition time intervals, which values may be used accordingly for motion detection (e.g., even specific to acquisition time intervals). Example embodiments in which measurement values are acquired for all radiofrequency pulses of the magnetic resonance sequence, and consequently of the acquisition protocol, are also beneficial since then, motion effects may be tracked over the entire time interval and the like. For example, it is therefore possible also to identify individual k-space trajectory sections sampled in an acquisition time interval (e.g., k-space lines), as affected by motion.

A selection of specific radiofrequency pulses is beneficial, for example, when a variant of the method according to the present embodiments is chosen in which, considered individually, measurements are taken for maximally similar or even identical radiofrequency pulses in order already in this way to bring about an outstanding comparability of the measurement values or of measurement value groups (e.g., partial time series) of the time series.

Thus, a beneficial development of the present embodiments provides that all the radiofrequency pulses for which at least one measurement value is acquired have at least one identical characteristic variable. In this variant, radiofrequency pulses may therefore be selected or marked such that radiofrequency pulses that are identical in at least one characteristic variable are selected. This simplifies a correction in the preprocessing step since the radiofrequency pulses are already basically comparable. Characteristic variables, generally speaking, may include a flip angle and/or a pulse amplitude and/or a pulse phase and/or a pulse frequency and/or a pulse shape described by the radiofrequency pulse.

For example, it may be provided, in a first variant, as already mentioned, to use radiofrequency pulses that are so similar that a pulse-specific cleaning measure is not necessary, but instead, fewer correction measures requiring parameters in the background information may be used, as will be explained in more detail in the following. For example, it may be sufficient as background information that, with regard to specific sections or to partial time series of the time series generally, certain profiles that may be removed (e.g., by detrending) result. In one embodiment, in this context, the radiofrequency pulses for which at least one measurement value is acquired correspond to one another, at least in their flip angle (e.g., in addition also in their pulse shape). In an example of a TSE sequence as the magnetic resonance sequence, refocusing radiofrequency pulses in the echo trains that relate to an identical flip angle are marked for the acquisition of at least one measurement value, for example.

Since, however, for example, with regard to the flip angle, varying flip angles or other varying characteristic variables may frequently occur within magnetic resonance sequences and consequently also acquisition protocols, a beneficial development of the present embodiments in a second variant may also provide a pulse-specific correction. For example, it may be provided that the background information describes a characteristic variable differing between different radiofrequency pulses for which a measurement value is acquired. The idea in this embodiment is therefore to correct measurement values using prior knowledge about the respectively concurrently played-out radiofrequency pulse. In this context, suitable characteristic variables in which the radiofrequency pulses may differ include, for example, the pulse frequency, the pulse phase, the flip angle (e.g., pulse amplitude or complex pulse integral values over the course of the radiofrequency pulse), and/or the pulse shape.

In this case, the at least one characteristic variable in which the radiofrequency pulses are different from one another may be determined, for example, from control information for generating the radiofrequency pulse. If the transmission chain is actuated, for example, based on data objects provided as control information, the at least one characteristic variable may be derived from these data objects for the corresponding radiofrequency pulse in each case (e.g., already at the time of its output). In other words, prior to or during the radiofrequency pulse being played out (e.g., a number of milliseconds before), commissioned real-time events may be supplied to the measuring device and/or to the measurement electronics and/or to the control device (e.g., to a measurement unit and/or a preprocessing unit, such as by a function known as a real-time event listener (RT event listener)). For example, as control information in addition to data objects related to the pulse shape of the radiofrequency pulse and characterizing a real-time event, pulse frequency-pulse phase events may be processed as further data objects since the played-out radiofrequency pulses and consequently their characteristic variables are generated from the combination of these real-time events. The complex-value envelope of the radiofrequency pulse and the carrier signal, parameterized with pulse frequency and pulse phase, result in the overall radiofrequency pulse such that any characteristic variables may be derived herefrom.

In the method according to the present embodiments, it is, for example, also possible as well as beneficial if at least the portion of the preprocessing step relating to the at least one characteristic variable is performed for each measurement value (e.g., immediately after its acquisition). Ultimately, therefore, a real-time correction is possible, which may take place, for example, already before characteristic values of the time series are stored for subsequent evaluation. This real-time preprocessing, which may therefore take place in relation to each individual radiofrequency pulse (e.g., is pulse-specific) may be realized with little overhead both using software and using hardware, or by a combination of software and hardware. For example, the control information (e.g., data objects marking real-time events) may be queried immediately and used for correction with regard to the at least one characteristic variable in which radiofrequency pulses are different from one another.

In this case, it may be provided, for example, that the measurement values are normalized in the preprocessing step with respect to one or more of the at least one measurement variable (e.g., a measurement variable describing the flip angle). For example, measurement values for radiofrequency pulses having different pulse amplitudes or generally flip angles and/or pulse phases may be made comparable in this way, with the result that such a correction in the preprocessing step allows a comparison of radiofrequency pulses having different properties. If, for example, the flip angle varies across the radiofrequency pulses, a scaling of the measurement value may be provided such that a normalization to a specific flip angle value is performed. This enables motion information to be reliably and robustly determined also for acquisition protocols having many variable flip angles, for example, in the case of TSE sequences, also for echo trains in which the flip angle varies. This is the case, for example, with Sampling Perfection with Application optimized Contrast using different flip angle Evolution (SPACE) and hyperecho sequences. If the pulse shapes, for example, as refocusing radiofrequency pulses (e.g., as well as possibly other characteristic variables) are identical, it may be sufficient already, in order to achieve a significant improvement in the identification of motion, to normalize the acquired measurement values to the different flip angle (e.g., by scaling). The same applies analogously to other characteristic variables. However, example embodiments in which a normalization is performed for a plurality of characteristic variables may be provided.

In addition or alternatively for this second variant, a beneficial development may provide that characteristics information is used that assigns to characteristic variables correction values that describe deviations occurring due to different values of the at least one characteristic variable on account of the characteristics of the transmission chain used to output the radiofrequency pulses. The correction value is used for correcting the measurement values with respect to the deviations. In this way, characteristics of the transmission chain (e.g., frequency-dependent attenuation behavior) may also be incorporated into the correction in the preprocessing step. If the behavior of the transmission chain in relation to one or more characteristic variables is known, the measurement values may be corrected according to the at least one characteristic variable in order to improve the comparability or the evaluation capability generally. In practice, the characteristics may include a transmission behavior (e.g., a frequency-dependent attenuation behavior of the transmission chain). For example, the characteristics information may therefore include a characteristic map and/or a lookup table and/or a mathematical relationship and/or a learned relationship (e.g., in the form of a trained association function) in order to determine correction values for specific values of the at least one characteristic variable. The characteristics (e.g., the transmission behavior) may have been determined previously, for example, by measurements or the like. For example, the characteristics information may also have been determined empirically.

The already mentioned frequency-dependent attenuation behavior occurs particularly frequently in transmission chains in magnetic resonance devices, with the result, for example, that a different attenuation occurs at different pulse frequencies. This is relevant, for example, when the acquisition protocol relates to an acquisition in multiple slices since a different pulse frequency for each of these slices is required for slice-selective excitation when a gradient is present in the slice selection direction. The different attenuation effect then occurring may be cleaned, at least to some extent, by corresponding characteristics information that assigns suitable correction values (e.g., correction factors for scaling) to the pulse frequencies.

To sum up, it is possible in this second variant, by the correction of the measurement values as B1 reflection raw data using prior knowledge about the respectively played-out radiofrequency pulse, to reduce influencing factors on the time series, such as slice position, flip angle, and pulse shape. This facilitates the data analysis during the determination of the motion information and, for example, permits its application to echo trains having variable flip angles and/or different preparation modules.

Regardless of whether comparable radiofrequency pulses are used with respect to at least one characteristic variable without a pulse-specific correction (e.g., first variant) or whether a pulse-specific correction is performed on account of at least one characteristic variable (e.g., second variant), a correction relating to multiple measurement values and removing an expected property of the time series, at least to some extent, may also be performed in the preprocessing step. This, in the case of the first variant or of an unconsidered influence of a characteristic variable or non-covered effects in the case of the second variant, permits an improvement in evaluability by removing non-motion-related properties of the time series. In other words, an effective removal of disrupting influencing factors in the time series may nonetheless be accomplished in the first variant by a correction relating to multiple measurement values using fewer parameters, while in the second variant, effects remaining after the pulse-specific correction may be corrected.

In practice, corresponding embodiments of the method according to the present embodiments may provide that in the preprocessing step the time series is subdivided initially based on at least one piece of time structure information of the at least one piece of background information into partial time series having a property expected for the respective partial time series, whereafter the cleaning is performed in a process specific to each partial time series. It is therefore proposed to subdivide the time series retrospectively (e.g., following the acquisition of multiple measurement values) into partial time series that reveal a specific profile property (e.g., therefore a particular expected behavior that is independent of a movement that has possibly occurred and is therefore to be removed). To that end, the measurement values are grouped into related sections, where in practice, it may be provided that the at least one piece of time structure information is chosen from the group including a subdivision of the acquisition protocol into repetitions, a subdivision of the acquisition protocol into echo trains, a subdivision of the acquisition protocol into sections containing slice groups having slices that are acquired immediately contiguously, and a subdivision of the acquisition protocol into acquisition procedures for at least one slice.

In this way, therefore, effects that reveal themselves in subgroups (e.g., partial time series) of the time series may be handled in a targeted and robust manner in the preprocessing step. This relates, for example, to properties of the time series that manifest themselves in slice-specific measurement values, echo-train-specific measurement values, repetition-specific measurement values, and/or section-specific measurement values. In order to form partial time series, the measurement values may therefore be sorted according to measurement values for the slices, measurement values for echo trains, and the like.

This shall be explained in more detail with the aid of an example. If, for example, magnetic resonance data is acquired using a TSE sequence from twenty-seven slices in eleven repetitions at an echo train length of sixteen, measurement values may be acquired for all the refocusing radiofrequency pulses such that a representative measurement value may be determined, for example, for each acquisition time interval. The time series may then include 4752 representative measurement values, for example. Given a definition of partial time series according to repetitions, eleven partial time series, each having 297 (e.g., representative) measurement values, are therefore produced, while a definition according to slices results in twenty-seven partial time series, each having 176 measurement values. If an expected profile according to the background information is known within the partial time series, these may then be at least partially cleaned of this. Partial time series may also be defined according to different criteria (e.g., “dimensions”) and preprocessed separately in each case. For example, a preprocessing may be performed not only along a repetition but also, in addition, along slices and/or echo trains. However, a preprocessing may be preprocessed only in one subdivision dimension or a further splitting into partial time series (e.g., along echoes per echo train, slices, and repetitions).

A subdivision according to slice groups is beneficial, for example, when an interleaved acquisition of slice groups is performed. For example, a subdivision into slice groups having slices that are to be acquired consecutively, each offset from one another, may be provided in the acquisition protocol, for example, in order to avoid slice crosstalk. The slices within each slice group are acquired according to an order corresponding, for example, to a spatial direction. In one example embodiment, the slices may be numbered in an order along the slice selection direction. A slice group (e.g., one to be acquired first) is formed by the slices with odd numbers, and a further slice group is formed by the slices with even numbers. In such an offset (e.g., “interleaved”) acquisition with two slice groups, it has been shown that the number of similar temporal profile sections in the time series may also be doubled (e.g., a repetition having a similar profile property being present for each repetition relating to the slice groups sequentially). In the above-cited example of eleven repetitions, therefore, twenty-two characteristic sections may result in such a case. A subdivision according to these sections may then be performed.

The time structure information may include desired profile information (e.g., the same for each partial time series). The partial time series is corrected at least to some extent by a profile property described by the desired profile information. In this case, the desired profile information may indicate, for example, a functional profile that the measurement values within the time series take as expected according to the profile property. For example, the desired profile information may therefore indicate that a linear, square, or other measurement value profile is expected. In one embodiment, at least one detrending may be performed in order to correct the profile property. In this way, a trend in data (e.g., the partial time series) may be removed using known methods. Generally known procedures may be used (e.g., detrending by differencing, detrending by fitting to a model, and the like). Trials have shown that linear trends may frequently occur, as may be described by the desired profile information, such that a linear detrending may be used. However, a linear detrending has also already shown itself to be useful for other cases.

In practice, as already indicated above, it may be provided that the detrending is performed for multiple different subdivisions into partial time series (e.g., at least with respect to individual echo trains or slice groups and with respect to individual slices and/or echoes within an echo train and/or repetitions). In other words, a, for example, linear detrending may be performed, for example, both along echo trains and along slices, for which purpose corresponding partial time series are defined in each case. Thus, effects in relation to different partial time series may be handled, and the cleaning, and therefore evaluability, is improved.

With regard to the evaluation (e.g., the determination of the motion information), it may be provided that the motion information is determined at least to some extent by a comparison of measurement value groups of the time series with one another and/or with reference information. In this case, measurement value groups may also correspond to partial time series, such that a subdivision may be taken over from the preprocessing step. For example, a comparison for echo trains, for k-space trajectory sections for slices, sections, and the like, may therefore be performed. Excellent comparability is established thanks to the correction carried out in the preprocessing step such that movements may be reliably and robustly identified already at the reciprocal comparison stage, and corresponding motion information may be determined. Reference information may also be used since a type of “normalization” is performed as a result of the preprocessing step. Reference information may correspond to a predefined expected value or expectation profile without movement (e.g., as a threshold value). Such a threshold value, indicating the occurrence of motion, for example, may also be obtained from the time series or from a measurement value group itself (e.g., as a result of the preprocessing). In an actual embodiment variant, the threshold value may be determined, for example, as 1.5 to 4 times an average of the measurement values (e.g., of a median). However, the reference information may also be derived specifically for an acquisition procedure from sections in which it is certain no movement was present.

For example, with regard to a controlling of the acquisition procedure itself, it is beneficial if at least one comparison is performed already during the acquisition using the acquisition protocol. For example, a comparison may be conducted whenever comparable measurement value groups (e.g., partial time series) are present. For example, in an embodiment, it may be provided in this case that a sliding time window, ending at the current time point and recording multiple measurement values, is used. However, it is also possible to compare individual measurement values with one another and/or with reference information (e.g., a threshold value). It is, however, beneficial if the motion information is determined in principle based on a plurality of measurement values in order not to overestimate individual measurement errors/outliers. If measurement values representative of measurement intervals are used, this already amounts to an averaging, and it may make sense to determine whether motion is present or not also for individual values of such representative measurement values. In any case, an extremely fast detection of movement is possible.

A particularly beneficial development of the present embodiments provides that in the case of an acquisition in multiple slices, the motion information is determined on a slice-specific basis. The measurement value groups are therefore formed as measurement values all referred to a specific slice. Thus, for example, it is possible to determine portions to be discarded (e.g., individual k-space lines) on a slice-specific basis (e.g., therefore, per slice k-space). Generally speaking, therefore, it may be provided, as already discussed, that individual k-space sections (e.g., k-space lines) may be identified by the motion information as affected by movement.

As already mentioned, the present embodiments (e.g., improving the basis for the evaluation in the preprocessing step) opens the door to a number of potential applications.

Thus, it may be provided, for example, that at least one measurement value is acquired for at least one radiofrequency pulse without following acquisition time interval in a preprocessing time interval (e.g., in order to establish a steady state). The acquisition of magnetic resonance data is triggered if the motion information indicates no current movement. Therefore, radiofrequency pulses may be played out, for example, in order to hold a steady state. The acquisition of actual magnetic resonance data is performed only when no further movement is detected. An approach of this kind may prove advantageous, for example, in the case of GRE sequences as the magnetic resonance sequence.

In addition or alternatively, it may be provided that in the case of magnetic resonance data that is to be discarded due to a movement during the acquisition from a time period still continuing into the future, the pulse amplitude of further radiofrequency pulses (e.g., the transmit voltage used for their generation) is reduced in the time period. If, for example, motion is detected during a TSE echo train and it is clear that the magnetic resonance data of the echo train is to be discarded, the transmit voltage may be regulated downward for subsequent radiofrequency pulses in the remainder of the echo train in order to keep the radiofrequency irradiation into the patient to a minimum. The adjustment of the pulse amplitude is beneficially taken into consideration in further preprocessing steps for the measurement values of the radiofrequency pulses for which the fitting was performed. This provides, on account of the preprocessing step provided anyway, that further measurement values may nonetheless be acquired and interpreted in order, for example, to establish whether and when the movement is terminated. In this context, a further beneficial development of the present embodiments may provide that in the case of magnetic resonance data acquired during the reduction of the pulse amplitude but nonetheless to be used on account of the cessation of the movement, the data is corrected according to the reduction. Since the measure is known, it may also be possible, at least in some cases, to correct and use acquired magnetic resonance data nonetheless.

In a particularly beneficial development of the present embodiments, it may be provided that the motion information for excluding magnetic resonance data acquired in at least one time period affected by movement is used for the reconstruction and/or for the reacquisition of the data. It is therefore possible, for example, to reacquire magnetic resonance data previously corrupted by movement. In the example of a TSE sequence, a reacquisition echo train that, for example, may contain k-space trajectory sections rendered unusable due to movement (e.g., instead compulsorily of such that previously belonged to an echo train) may then be added. If no magnetic resonance data was affected in individual segments or if all other data has already been recorded, a beneficial development of the present embodiments may provide that the k-space center is redundantly sampled a further time in the reacquisition echo train. In other words, the reacquisition echo train may be filled by further samplings of k-space regions to be acquired redundantly. The signal-to-noise ratio may be increased in this way.

However, it may be provided, at least for a portion of magnetic resonance data affected by movement, to dispense with a reacquisition and to perform a reconstruction based on incomplete magnetic resonance data. In this context, a beneficial development of the present embodiments provides that a (e.g., trained) reconstruction function compensating for missing magnetic resonance data may be used for the reconstruction (e.g., a deep resolve boost function based on an unrolled network architecture). Techniques based on machine learning have already been proposed in the prior art in order to achieve a high-quality reconstruction even in the case of magnetic resonance data present that is not complete. These techniques may also be beneficially used within the scope of the present embodiments in order to avoid, at least to some extent, a reacquisition of magnetic resonance data affected by movement.

In a further embodiment (e.g., with motion information determined in a dedicated manner), it may be provided that the motion information and/or the measurement values be transferred as additional input data to a trained reconstruction function. In this way, a reconstruction function may therefore learn which magnetic resonance data may be affected by movement (e.g., how strongly) and use this knowledge in the reconstruction of a magnetic resonance image dataset. In one embodiment, the motion information is used for preconditioning at least one correction function used for the reconstruction and/or for preparing the latter. A motion correction may therefore be improved by using the motion information derived robustly, reliably, and with high quality from the preprocessed time series. Possible retrospective correction methods of this type include Scout Accelerated Motion Estimation and Reduction (SAMER) (cf., D. Polak et al., MRM 87 (2022), pages 163-178) and Targeted Motion Estimation and Reduction (TAMER) (cf., M. W. Haskell et al., IEEE Trans Med Imaging 37(2018), pages 1253-1265). The motion information may be used, for example, in order to select k-space trajectory sections (e.g., k-space lines) relevant to the correction or to make a decision about whether it is even necessary to make a correction.

It may further be provided that the motion information is evaluated during the acquisition procedure using the acquisition protocol for the output of at least one piece of information to an operator and/or for changing the acquisition protocol. In this way, an operator may be notified of occurring movement (e.g., promptly) and respond accordingly (e.g., by initiating countermeasures during the acquisition procedure and/or by acting on the patient, such as on account of a corresponding warning as information). It is further conceivable to initiate an automatic response by automatically fitting the acquisition protocol during the acquisition procedure (e.g., in the case of regular, small movements) to an acquisition scheme that is more robust with regard to movements and/or to a magnetic resonance sequence better suited to occurring movements.

For example, it may be provided in this context that the motion information and/or the measurement values of the time series be used as additional input data for an image quality assessing (e.g., supervising) trained image quality function (e.g., a neural network).

In general, a trained function maps cognitive functions that human beings associate with other human brains. By training based on training data (e.g., machine learning), the trained function has the ability to adapt to new circumstances and to detect and extrapolate patterns.

Generally speaking, parameters of a trained function may be adapted by training. For example, supervised learning, semi-supervised learning, unsupervised learning, reinforcement learning, and/or active learning may be used. In addition, representation learning (also known as “feature learning”) may also be used. The parameters of the trained function may be adjusted (e.g., iteratively) by multiple training steps.

A trained function may, for example, include a neural network, a support vector machine (SVM), a decision tree, and/or a Bayesian network, and/or the trained function may be based on k-means clustering, Q-learning, genetic algorithms, and/or association rules. For example, a neural network may be a deep neural network, a convolutional neural network (CNN), or a deep CNN. Further, the neural network may be an adversarial network, a deep adversarial network, and/or a generative adversarial network (GAN).

With regard to a change to the acquisition protocol or generally a controlling of the acquisition procedure, a development provides that in order to control the acquisition procedure, the motion information is determined during the acquisition using the acquisition protocol and is evaluated by at least one measure condition that is met if a strength threshold value is exceeded by the strength of the current movement described by the motion information. Each measure condition is assigned a control measure that is actioned if the measure condition is met. This is based on the idea that although in general no determination of more accurate motion parameters is possible using the motion information (e.g., in terms of type, direction, and strength of the movement), it is possible to reliably detect relevant, current movement that therefore has a strength determining the relevance. For example, the motion information may in this case, as already described above, be determined by performing the at least one comparison already during the acquisition using the acquisition protocol. There is therefore a quasi-continuous signal present with regard to relevant current movements during the acquisition.

This quasi-continuous signal (e.g., the motion information) may now be used to trigger actions that intervene in the execution sequence of the acquisition protocol (e.g., change this itself or its timing). These actions are described by control measures that are assigned to respective measure conditions that evaluate the motion information. In this way, it is possible, by acting in a timely manner, to reduce the frequency of a need for reacquisition procedures and to increase the image quality. Further, the acquisition time is not extended, at least when no movement occurs.

In a development, it may be provided in this context that the control measure relates for at least one of the at least one measure conditions to a fitting of the acquisition protocol for the next possible acquisition of a navigator dataset and/or of a reference dataset for a parallel imaging procedure and/or for an undersampling. A movement may lead to a change in the patient state (e.g., in positions and orientations in the acquisition region) compared to the original patient state. For example, a short intake of breath prior to repeating a breathhold may lead to a different respiratory state or a change in position that may compromise the mutual compatibility of the magnetic resonance data, but also the handling thereof during the reconstruction. In parallel imaging, for example, kernels fitted using calibration parameters that are based on the patient state at the time of the acquisition of a reference dataset are used to separate magnetic resonance data. Analogously, during undersampling, data may be supplemented based on such calibration parameters in the reconstruction, or these processes may even be combined. Examples of imaging techniques in which such reference scans are conducted for the acquisition of reference datasets include Generalized Autocalibrating Partially Parallel Acquisitions (GRAPPA), Simultaneous Multi Slice (SMS), SENSE, Compressed Sensing (CS), and deep resolve measurements.

In practice, it may be provided that the navigator dataset is evaluated for supplementing the motion information and/or for determining supplementary information with respect to a quantification of the current movement and/or a new patient state following the movement. For example, a motion field starting from the last navigator measurement may be determined. Such evaluation results may be used, as is generally known, for motion compensation of the magnetic resonance data. The great advantage of the proposed approach is, for example, that no further regular acquisitions of navigator datasets are necessary, but this is only to take place when movement also actually occurred.

With regard to the reference dataset, it may be provided that this is evaluated for determining calibration parameters for a reconstruction of the magnetic resonance data acquired after the current movement that led to its acquisition. This, too, is generally known in the prior art. Particular advantages result here, for example, in SMS diffusion imaging since calibration errors in submaps may lead to errors in the definitive diffusion map. The errors are not identifiable or identifiable only with difficulty. Such errors may be avoided by responding directly to movement that has occurred.

During the acquisition of navigator and/or reference datasets, it is advantageous if, when holding a state of equilibrium (e.g., steady state) by outputting radiofrequency pulses with following acquisition time intervals according to a timing sequence scheme (e.g., sequence timing), the timing sequence scheme is also maintained for the acquisition of the navigator dataset and/or the reference dataset. Steady-state effects may be avoided in this way. For example, when sufficiently strong current movement has been detected in an echo train, a next echo train may be inserted in which the k-space center is sampled in order to form a navigator, the timing sequence scheme (e.g., sequence timing) of which remains unchanged. For example, in the case of a spin echo sequence (SE) or a gradient echo sequence, a simple replanning of the k-space points to be sampled (e.g., k-space lines) may be sufficient.

Analogously, the use of the same timing sequence scheme as for the magnetic resonance data is also possible without problems in the case of calibration datasets.

In one embodiment, in a multislice acquisition and when a prior navigator dataset describing the original motion state and relating to all the slices and/or a prior calibration dataset is present, the navigator dataset and/or the reference dataset may be acquired only for one of the multiple slices. The respective prior dataset therefore serves as a reference by which the newly acquired navigator or the newly acquired reference may be aligned. However, it is also conceivable to acquire all of the slices in order to achieve a maximally accurate three-dimensional coverage.

When at least one preparation module having at least one preparation pulse is used, a development of the method provides that the control measure of one of the at least one measure conditions relates to a repeated output of the preparation module. Such a preparation module may relate, for example, to imaging techniques such as Magnetization Transfer (MT), Fluid Attenuated Inversion Recovery (FLAIR), and/or Arterial Spin Labeling (ASL). The repeated output provides that the preparation with respect to the acquired magnetic resonance data also relates to the right spins. An erroneous acquisition of subsequent magnetic resonance data is thus avoided.

A beneficial development may further provide that for one or more of the at least one measure condition, the control measure relates to a halting of the acquisition protocol. In this case, a pausing may be provided, which provides that the acquisition in accordance with the acquisition protocol may be resumed (e.g., following termination of the movement). For this purpose, it may be provided, as already explained above for the commencement of the acquisition using the acquisition protocol, that at least one measurement value is acquired for at least one radiofrequency pulse without a following acquisition time interval in a continuation interval (e.g., in order to restore a steady state). The acquisition of magnetic resonance data is resumed when the motion information no longer indicates any current movement. For example, a coughing attack may be followed by a wait for a new breathhold. In the case of such a continuation, it is particularly beneficial if initially a navigator dataset and/or a calibration dataset, as described above, are/is acquired as a further control measure with the continuation since the patient may be situated in a different patient state (e.g., a different position and/or orientation, such as in relation to breathing). This allows a swift and beneficial response to unforeseen events.

In addition to the method, the present embodiments also relate to a magnetic resonance device having a main magnet unit containing a main magnet for generating a main magnetic field, a gradient coil array, and a radiofrequency coil array, to which there are assigned: a transmission chain for outputting radiofrequency pulses during an acquisition procedure in accordance with an acquisition protocol that uses at least one magnetic resonance sequence in an acquisition period and includes radiofrequency pulses that precede acquisition time intervals; and a measuring device for measuring a reflected power reflected back by a coil element of the radiofrequency coil array that is used to output the radiofrequency pulses. The magnetic resonance device further includes a control device that has: a sequence unit for controlling the acquisition procedure in accordance with the acquisition protocol and for actuating the measuring device for the acquisition of at least one measurement value of the back-reflected reflected power for at least some of the radiofrequency pulses in each case in a measurement interval, such that a time series of measurement values is produced over the acquisition period; an evaluation unit for evaluating the time series of measurement values in an evaluation step for the purpose of detecting an occurring movement of the scanned examination subject, the movement being described by motion information, where the sequence unit is configured to use the motion information for controlling the acquisition procedure, and/or a reconstruction unit is provided that is configured to use the motion information in a reconstruction of an image dataset from acquired magnetic resonance data; and a preprocessing unit that is configured to clean the time series of the property, at least to some extent, in a preprocessing step prior to the evaluation step using at least one piece of background information that describes an expected property of the time series that is not motion-related.

All statements made with respect to the method according to the present embodiments may be applied analogously to the magnetic resonance device according to the present embodiments, and vice versa, such that the same advantages may be achieved.

The control device may include at least one processor and at least one storage device. Functional units for performing steps of the method according to the present embodiments are formed by hardware and/or software (e.g., in the present case, at least a sequence unit, an evaluation unit, and a preprocessing unit, where a reconstruction unit is also generally known for control devices of magnetic resonance devices and accordingly is also usefully provided according to the present embodiments). Further functional units may likewise be provided (e.g., in view of the different proposed applications). For example, a control unit for actuating further components of the magnetic resonance device may be present. In this case, the sequence unit corresponds to generally known sequence units for control devices of magnetic resonance devices that control the acquisition operation and in the present case also has the means to actuate the measuring device for the acquisition of at least one measurement value of the time series (e.g., for correspondingly marked radiofrequency pulses). The measuring device may be formed in this case by generally present components (e.g., a directional coupler and/or a measurement electronics device).

A computer program according to the present embodiments may be loaded directly into a storage device of a control device of a magnetic resonance device and has program means such that when the computer program is executed in the control device, it causes the latter to perform the steps of a method according to the present embodiments. The computer program may be stored on an electronically readable data medium according to the present embodiments, which therefore has control information stored thereon that includes at least one computer program according to the present embodiments and is configured such that when the data medium is used in a control device of a magnetic resonance device, the control device is configured to perform a method according to the present embodiments. The data medium is, for example, a non-transitory data medium (e.g., a non-transitory computer-readable storage medium, such as a CD-ROM).

### BRIEF DESCRIPTION OF THE DRAWINGS

FIG. 1 shows a general flowchart of example embodiments of a method;

FIG. 2 shows an example extract from an echo train of a TSE sequence;

FIG. 3 shows example profiles of measurement values of time series of different acquisition procedures;

FIG. 4 shows a first profile following a cleaning operation in a preprocessing step;

FIG. 5 shows a second profile following a cleaning operation in the preprocessing step;

FIG. 6 shows a third profile following a cleaning operation in the preprocessing step;

FIG. 7 shows a fourth profile following a cleaning operation in the preprocessing step;

FIG. 8 shows a comparison of measurement values with a scaled difference (offset) of a pulse frequency from a nominal Larmor frequency of a magnetic resonance device;

FIG. 9 shows a flowchart of a first possible sequence of substeps of the preprocessing step;

FIG. 10 shows a flowchart of a second possible sequence of substeps of the preprocessing step;

FIG. 11 shows a slice-specific profile of measurement values of the fourth profile for a first slice;

FIG. 12 shows a slice-specific profile of measurement values of the fourth profile for a second slice;

FIG. 13 shows a flowchart of substeps for using the motion information during the acquisition;

FIG. 14 shows a schematic diagram for triggering a navigator scan due to current movement;

FIG. 15 shows a schematic diagram of a magnetic resonance device according to the present embodiments; and

FIG. 16 shows the functional layout of the magnetic resonance device of FIG. 15.

### DETAILED DESCRIPTION

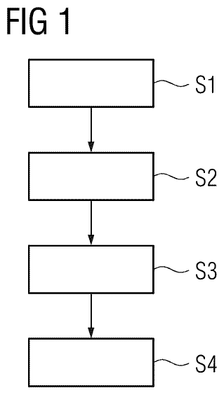

FIG. 1 shows a general flowchart of an example embodiment of a method. In the following, as a specific example, reference is made to its use in an acquisition procedure using an acquisition protocol in which a TSE sequence is used. Twenty-seven slices are acquired in eleven repetitions, where, in each repetition, initially, echo trains of an echo train length of sixteen for the slices with odd slice numbers of a series along the slice selection direction (e.g., slices one, three, . . . , twenty-seven) and thereafter for the slices with even slice numbers (e.g., slices two, four, . . . , twenty-six) are used in order to acquire magnetic resonance data (e.g., in the present case, one k-space line in each acquisition time interval following a refocusing radiofrequency pulse). These parameters and also the use of a TSE sequence are to be understood as purely by way of example.

According to act S1, measurement values are acquired for at least some of the radiofrequency pulses of the acquisition protocol in a measurement interval during the output of the radiofrequency pulse. The measurement values describe the reflected power reflected back by the at least one used coil element of the radiofrequency coil array of the magnetic resonance device that is used. For example, there may be provided in the supply line to the coil element a directional coupler by which the back-reflected reflected power may be measured by a measuring device that may also include a measurement electronics device based on the reverse voltage. A measuring device that may also measure the forward voltage (e.g., for SAR considerations) may be used.

In the present example, measurement values are acquired for every refocusing radiofrequency pulse immediately preceding an acquisition time interval. Example embodiments in which measurement values are acquired for each radiofrequency pulse or for other selected radiofrequency pulses may also be provided. The corresponding radiofrequency pulses are marked in the acquisition protocol so that a sequence unit may actuate the measuring device for the purpose of acquiring measurement values. Multiple measurements are taken during each measurement interval (e.g., 128 measurements) that are processed statistically so that at least one representative measurement value is produced for the measurement interval and consequently the acquisition time interval.

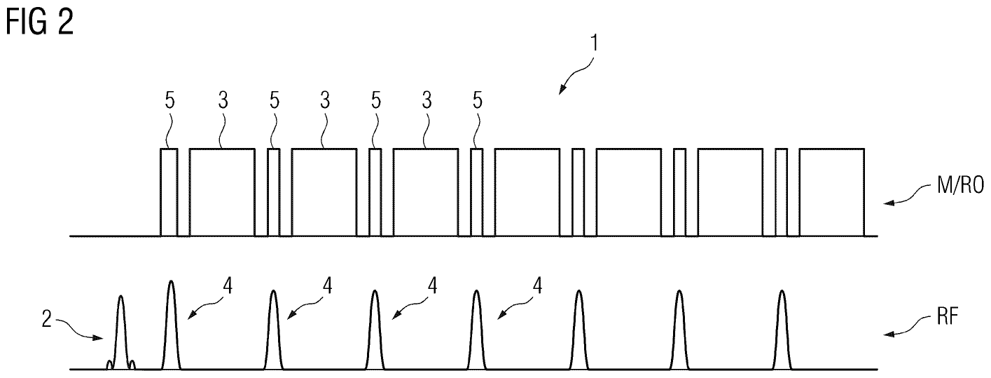

FIG. 2 shows an extract from the sequence diagram of an echo train 1, specifically its beginning. As shown, in the present specific example, no measurement value is acquired for the excitation radiofrequency pulse 2, although in addition to the acquisition time intervals 3 that follow the refocusing radiofrequency pulses 4, a measurement interval 5 is also provided for each refocusing radiofrequency pulse 4 for the purpose of acquiring the at least one measurement value. While the acquisition time interval 3 may also be referred to as RX-ADC on account of the open ADC, the measurement interval 5 then corresponds to a TX-ADC.

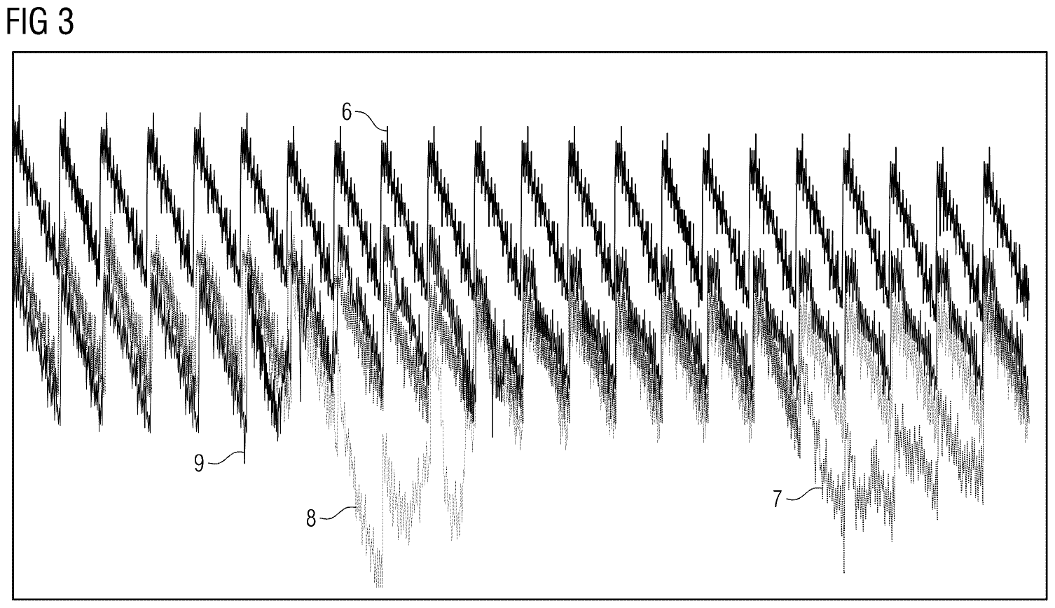

FIG. 3 shows possible time series 6, 7, 8, 9 (e.g., profiles) of measurement values for the specific example. Each of these time series 6, 7, 8, 9 is based on a different motion pattern. It is shown that in all the measurements, they consist of the eleven repetitions, where the twenty-seven slices were acquired in an interleaved manner, thereby resulting in twenty-two characteristic sections. If no motion takes place (e.g., compare the first time series 6), then the measurement value profile is similar for each characteristic section that is defined by a slice group. In the second profile, second time series 7, a movement is present toward the end, in the third profile, third time series 8, a movement is present in the center of the acquisition period. In the fourth time series 9, finally, two movements, short in time, specifically nodding of the head, occur.

Whereas, as shown, the longer lasting movements in the time series 6, 7, 8, 9 of FIG. 3, which may be referred to as B1 reflection raw data, may also be recognizable for the human observer as deviations, this is already more difficult to perceive for the time series 9 (e.g., brief movements). This is due to a plurality of further properties, not correlating with the movement, of the profiles of the measurement values, which may also make an automatic evaluation more difficult. It is therefore proposed in the first instance, prior to the evaluation in evaluation step S3 of FIG. 1, to perform a preprocessing based on background knowledge in a preprocessing step S2 in order to achieve a significant improvement in the identifiability of motion. In preprocessing step S2, background information that describes an expected, non-motion-related property of the time series 6, 7, 8, 9 is actually used in order to clean the time series 6, 7, 8, 9 at least to some extent of this property.

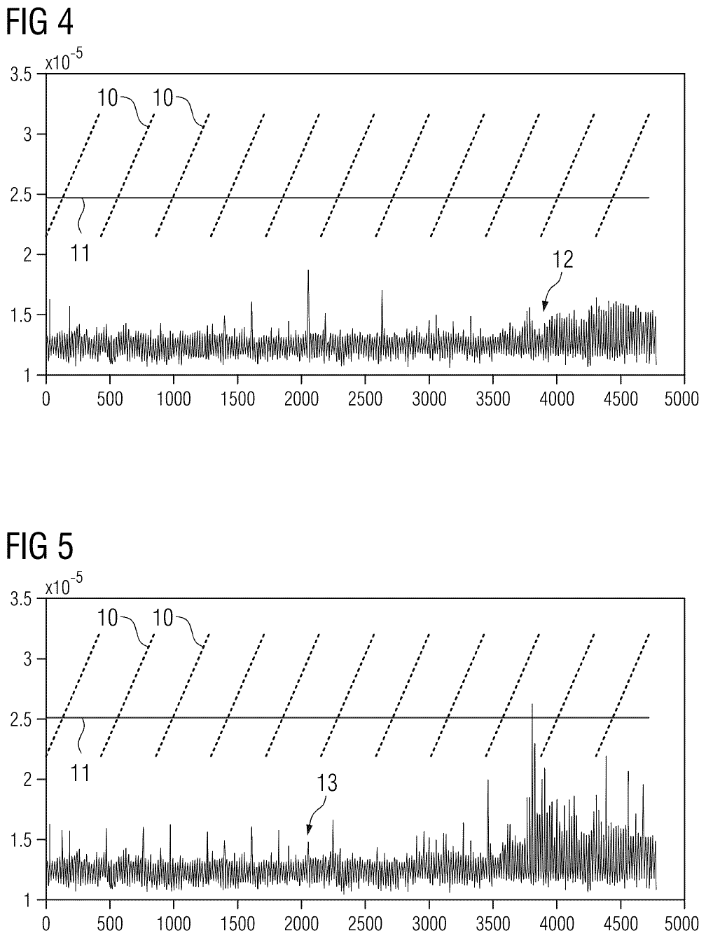

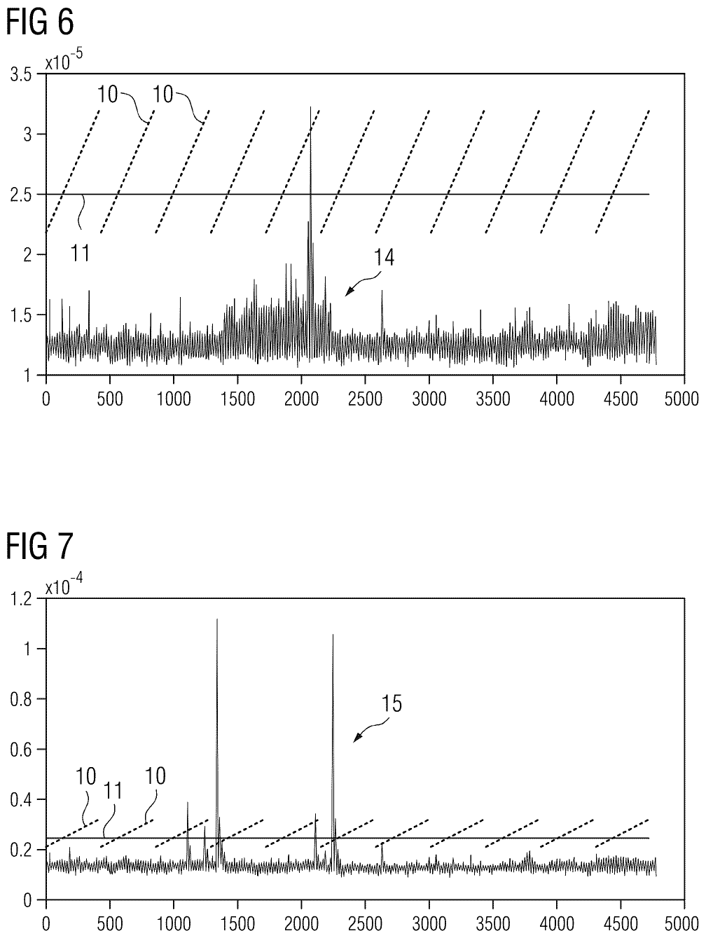

FIGS. 4 to 7 show example results of the preprocessing step S2 for the time series 6, 7, 8 and 9. In this case, as an aid to improved orientation, the eleven repetitions are indicated via rising ramps 10 in each case, the double of the median value of the (cleaned) measurement values (e.g., as potential threshold value for identifying relevant movements) being further shown by a line 11. FIG. 4 shows the cleaned time series 12 for the first time series 6, where it may now be clearly seen that no stronger deflections at all are present (e.g., no movement) and the measurement values remain at least substantially constant.

In the cleaned time series 13 for the second time series 7, the movement at the end of the acquisition period may clearly be seen, exactly as the movement in the center in the cleaned time series 14 for the third time series 8. In the cleaned time series 15 for the fourth time series 9, the short nodding movements are particularly clearly recognizable as significant deflections, whereas they were barely detectable in FIG. 3, time series 9.

The motion patterns may therefore be clearly recognized and identified in all cases. Compared to the original profile of the time series 6, 7, 8 and 9, for example, the time points of the movement taking place stand out more sharply in the cleaned time series 12, 13, 14 and 15. At this point, it is also conceivable to make the preprocessing parameters of the preprocessing step S2 adjustable in order to more strongly highlight different aspects of the movement that are to be detected (e.g., to determine the cleaned time series 12, 13, 14 and 15 specifically with regard to the evaluation taking place). Since the time points at which movement is present are relevant in the present case, the optimal removal of further, non-motion-related properties of the time series 6, 7, 8 and 9 may be provided and used further in the following.

Various options exist for the actual implementation of step S2. In one embodiment, the radiofrequency pulses coincide in one or, for example, even multiple characteristic variables to correct or clean only one property, describable via multiple parameters, of the profile of the measurement values or of partial time series. This makes sense, for example, when the refocusing radiofrequency pulses 4 all have the same pulse shape and relate to the same flip angle. Also possible, however, is a pulse-specific correction based on at least one characteristic variable of the radiofrequency pulse 9 (e.g., its flip angle and/or its pulse frequency and/or its pulse phase). Such a pulse-specific correction may then also be followed nonetheless by a profile correction via partial time series (e.g., a plurality of measurement values) in order to remove remaining effects.

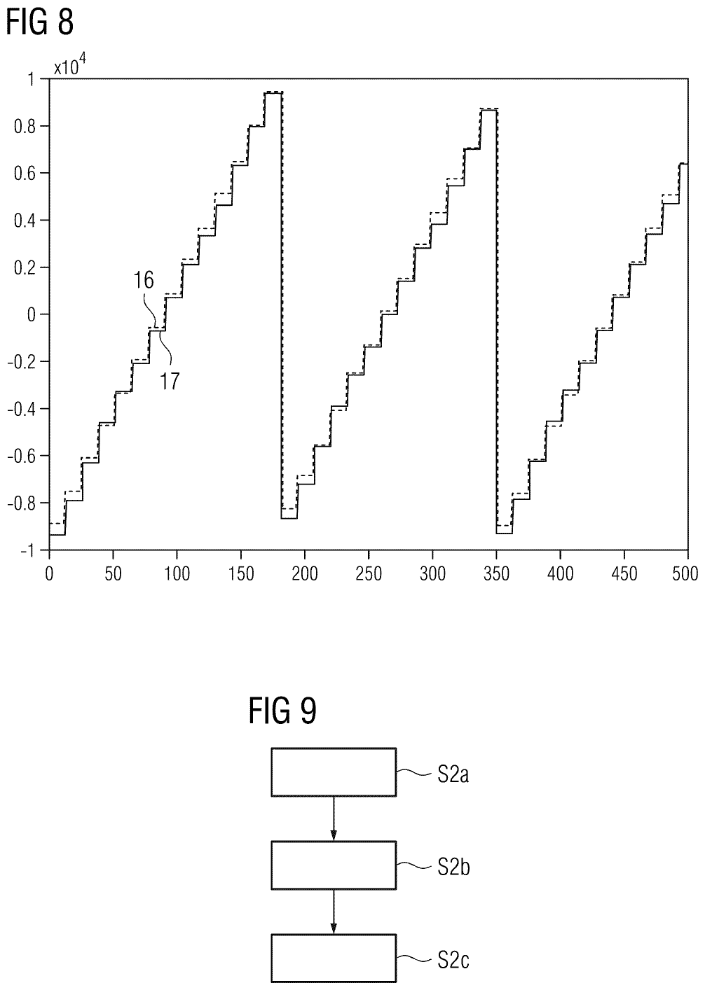

FIG. 8 shows in this regard, as an extract of an arbitrary time series, the profile 16 of the real part of the measurement values compared to the profile 17 of the frequency offset from the Larmor frequency for the respective radiofrequency pulses, the frequency offset having been suitably scaled. There is clearly a high degree of correlation that may be exploited to remove the dependence of the measurement values on the slice position. The pulse phase, which likewise varies depending on the slice position and may likewise be corrected, is not shown.

It may further be shown that the value profile of the measurement values during a measurement interval 5 reflects the profile of the radiofrequency pulse (e.g., both in terms of pulse frequency and of pulse phase). Both characteristic variables (and also further characteristic variables of the radiofrequency pulse) may easily be derived from control information to the transmission chain (e.g., data objects describing real-time events). This also applies, for example, to flip angles caused by radiofrequency pulses.

As the flowchart in FIG. 9 shows, the pulse-specific correction may be implemented in practice, for example, such that initially, in a step S2a, the at least one characteristic variable in relation to which the correction is to be applied is determined from control information in the corresponding measurement time intervals 5. For example, these may be the three cited characteristic variables flip angle, pulse phase, and pulse frequency.

In a step S2b, as the first correction step (e.g., immediately after the acquisition of a measurement value and prior to its being stored in the time series), a normalization is then performed with respect to at least one of the at least one characteristic variables determined in step S2a. This has proved to be beneficial, for example, at least with regard to the flip angle. Corresponding normalization factors may be determined empirically, for example, but also in some other way. For example, a scaling may be performed with regard to the flip angle. The normalization information that may be used for this forms a part of the background information.

If a normalization with regard to the flip angle is performed in this way, it becomes possible, for example, nonetheless to enable a useful evaluation in magnetic resonance sequences having many different flip angles.

In a step S2c, characteristics of the transmission chain may also be included as well (e.g., in the present case, frequency-dependent attenuation behavior). For this purpose, the background information includes characteristics information that assigns to the characteristic variables correction values that describe occurring deviations using different values of the at least one characteristic variable based on the characteristics of the transmission chain used to output the radiofrequency pulses. In the present case, a corresponding correction factor is assigned as characteristic variable for correcting a frequency-dependent attenuation behavior of the pulse frequency. The correction factor is applied to the measurement value in order to improve the comparability. Step S2c may also be performed immediately after the measurement, possibly before the measurement value is actually entered into the time series. A pulse-specific real-time correction is therefore provided.

It should further be noted at this point that this may also be implemented without difficulty and with relatively little overhead, whether it be in hardware or in software, owing to the control information, which is present anyway on the transmission chain.

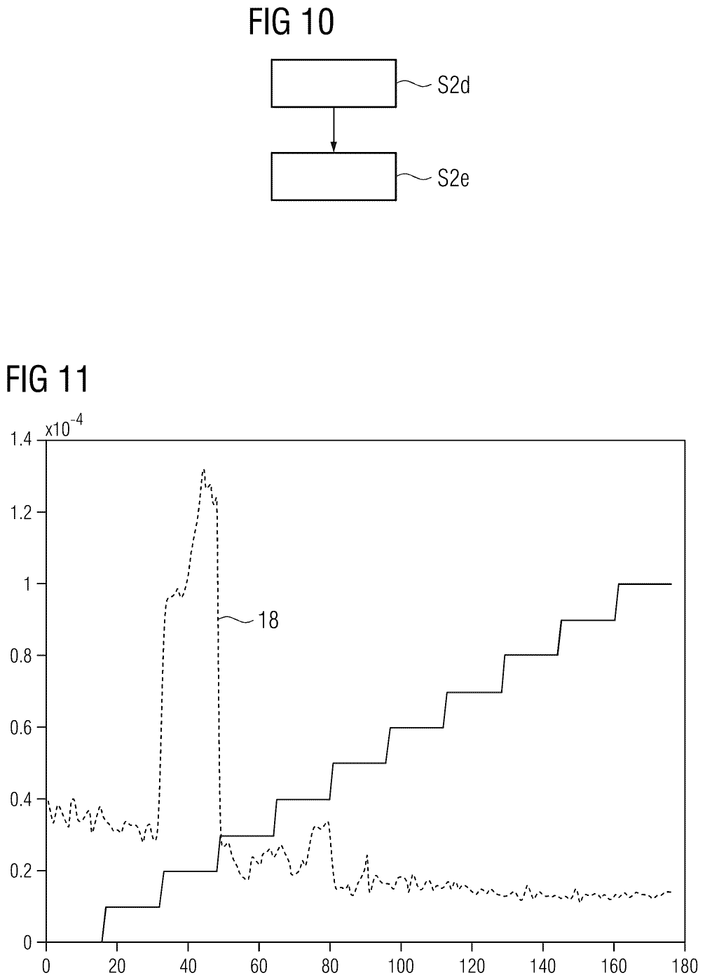

FIG. 10 shows a flowchart of substeps of step S2 in order to make corrections due to properties expected for partial time series. The part of the background information necessary for this is at least one piece of time structure information that also includes desired profile information.

In a step S2d, the measurement values are in this case grouped into related partial time series. This grouping may be accomplished according to repetitions, slices, characteristic sections (e.g., slice groups), echo trains, and the like. For example, for the characteristic sections of the slice groups with even and odd slice numbers of each repetition, it is clear from FIG. 3 that a roughly linearly falling trend is present. If a subdivision according to repetitions were to be performed, two such roughly linearly falling trends would be present for the slice groups. This expectation with respect to the profile of the measurement values within the partial time series is described by the desired profile information.

The subdivision into partial time series does not necessarily contain measurement values acquired immediately sequentially, but may also relate to measurement values acquired spaced apart in time (e.g., in the case of slices). Depending on the actual acquisition protocol or actual magnetic resonance sequence, there may also be trends present for such partial time series that may be described by the desired profile information.

In step S2e, the desired profile information is then used in order to clean, at least to some extent, the partial time series of the profile property expected for these. A detrending may be performed for this purpose. In the actual example, to which the illustrated graphs also relate, a linear detrending may be set, for example, for the characteristic sections. The linear detrending may be performed for several subdivision dimensions (e.g., both along the echo trains and along slices or shots). In one embodiment, a detrending for just one type of partial time series, but a subdivision according to several subdivision dimensions (e.g., echoes per echo train, slices, and repetitions) may be provided.

The actual time structure information for specific use cases may easily be determined empirically and/or analytically and may differ accordingly from use case to use case (e.g., from magnetic resonance sequence to magnetic resonance sequence or acquisition protocol to acquisition protocol).

Further, the correction according to steps S2d and S2e may also be implemented already prior to termination of the acquisition protocol (e.g., before the expiry of the acquisition period, such as always as soon as a partial time series is complete or contains sufficient measurement values).

Returning to FIG. 1, in an evaluation step S3, an evaluation of the respective currently acquired, cleaned time series 12, 13, 14, 15 is performed in order to determine motion information that is then used in a step S4 in order, if necessary, to influence the controlling of the acquisition procedure and/or the reconstruction of an image dataset from the acquired magnetic resonance data. Thus, the evaluation in evaluation step S3 may also take place at least in part already during the acquisition period. It is beneficial in this case to use a sliding time window that ends at the current measurement value. The evaluation may, for example, include comparisons of measurement value groups (e.g., also partial time series) with one another and/or with reference information (e.g., the already cited threshold value).

There exists a number of real-world options for using the motion information, determinable, for example, robustly, reliably, and with high quality in an improved manner, or the cleaned time series 12, 13, 14, 15, at least some of which are to be explained once more hereinbelow.

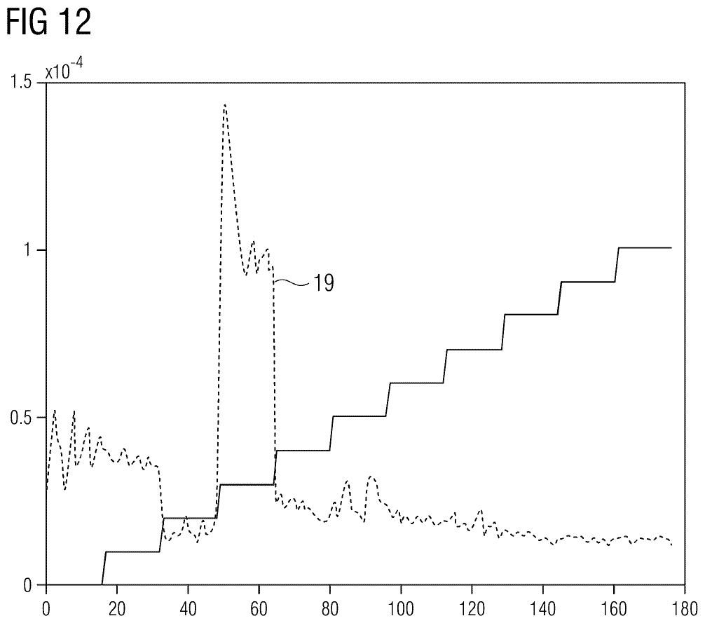

Measurement values may be classified into slice-specific measurement value groups if such measurement value groups are not already present anyway as partial time series. Profiles of the measurement values of the cleaned time series 12, 13, 14, 15 may therefore be formed and evaluated for each slice (e.g., by corresponding comparisons). This shall be explained in more detail once again for the cleaned time series 15 resulting from the fourth time series 9 with reference to FIGS. 11 and 12. In addition to the resulting measurement value groups (e.g., slice-specific profiles 18 for a first slice and 19 for a second slice), it is also indicated there, in the form of steps, the repetition from which the corresponding plotted measurement values originate. In the actual example, there are therefore sixteen measurement values (e.g., number of acquisition time intervals 3 in each echo train 1, such as echo train length) present for each step. It may very clearly be seen for both slices that certain echo trains 1 are affected by the first nod (e.g., the first brief movement). Whereas the third echo train 1 was affected in the first slice (FIG. 11), it is already the fourth echo train 1 in the second slice (FIG. 12). The two slices are only slightly affected by the second nod.

In a further refinement, it is even possible to identify individual k-space lines affected by movement. This permits the application thereof also for other magnetic resonance sequences, such as GRE sequences, for example.

Acquired magnetic resonance data may be identified as corrupted by movement based on the motion information. The magnetic resonance data may then, for example, be excluded from a reconstruction, where, for example, for the reconstruction in step S4, a trained reconstruction function (e.g., a deep resolve boost function) that is configured to compensate for missing magnetic resonance data is used.

In addition or alternatively, corrupt magnetic resonance data may be discarded, and the acquisition may be repeated upon completion or interpolated (e.g., in an additional reacquisition echo train or generally a reacquisition protocol section). In this process, it is then possible, if individual portions of the reacquisition protocol section are not required for the reacquisition of magnetic resonance data, to repeat an acquisition of the k-space center in order in this way to increase the signal-to-noise ratio.

It is also possible with the approach described here to achieve a triggered acquisition by initially, in order to hold a state of equilibrium (e.g., steady state), playing out radiofrequency pulses without a subsequent acquisition time interval 3 until the measurement values indicate that no further motion is detected. The acquisition of magnetic resonance data is then performed.

If motion is detected during a TSE echo train and it follows therefrom that the magnetic resonance data of the echo train is to be discarded, the transmit voltage for the radiofrequency pulses may be regulated downward for the remainder of the echo train in order to reduce the radiofrequency irradiation into the patient, and hence the SAR exposure, to a minimum. It is nonetheless possible to continue the acquisition of measurement values for the time series 6, 7, 8, 9 since a correction with respect to this reduced transmit voltage (e.g., characteristic variable flip angle: scaling/normalization) is possible within the scope of the cleaning process.

A combination with methods for assessing image quality may also be realized in that, for example, the motion information and/or the measurement values form additional input data for an image quality function. An evaluation with regard to an automatic control action of the acquisition procedure and/or the output of information to an operator may also be performed generally. For example, a warning may be output to an operator in the event of movement, or a switch may be made to a different acquisition scheme or a different magnetic resonance sequence.

It is further possible to use the motion information and/or the measurement values of the cleaned time series 12, 13, 14, 15 for preconditioning retrospective correction methods such as TAMER or SAMER, for example, in order to select k-space lines relevant to the correction or to reach a decision on whether a correction needs to be made.

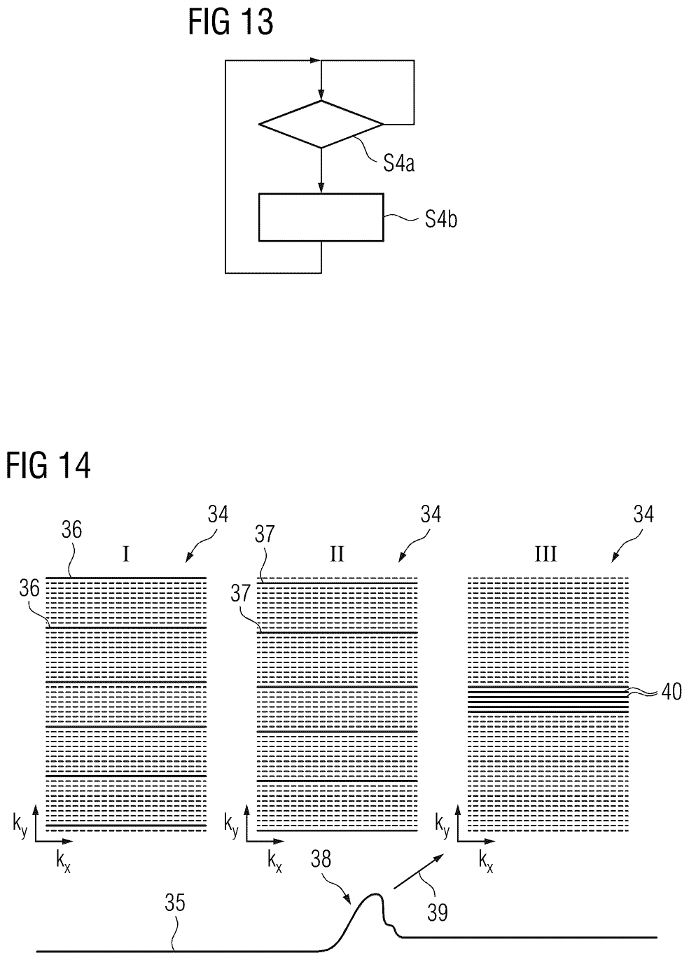

FIG. 13 shows a flowchart of an actual course of action with regard to a change of the acquisition protocol or generally a controlling of the acquisition procedure. Whenever updated motion information is available during the acquisition using the acquisition protocol, it is checked there in a step S4a whether at least one measure condition evaluating the motion information is met. The measure condition is assigned at least one control measure, which is actioned in a step S4b if the condition is met. The motion information is therefore evaluated as a quasi-continuous signal in order to trigger a control measure in the event of relevant motion (e.g., movement the strength of which exceeds a strength threshold value). The aim of the control measures is, for example, to compensate for certain effects of motion, though without prolonging (e.g., significantly) acquisitions with little or no movement. Use is made of the fact that the motion information may be provided without intervening in the time characteristic of the acquisition protocol.

FIG. 14 explains an actual example with the aid of a succession of echo trains I, II and III, which are symbolized based on the sampling in the k-space 34. An acquisition protocol including a TSE sequence is used in this case. The movement over time is indicated schematically by a signal curve 35. K-space lines 36, 37 are sampled in echo trains I and II. In echo train II, it is now established, see signal region 38, that strong current movement is present that is described by the motion information. A measure condition to which the acquisition of a navigator dataset is assigned as control measure is met.

Thus, compare arrow 39 in echo train III using the same timing sequence workflow as in echo trains I and II in order to maintain the steady state, a navigator dataset is acquired by sampling the k-space center. In the present case, this is possible by simple replanning of the k-space lines 40 that, as shown in FIG. 14, are all located in the central region of the k-space 34. Steady-state effects that may occur due to changed timing are therefore avoided, though a full navigator acquisition, which enables an accurate determination of motion parameters or at least of the new patient state, is performed. A correction of subsequently acquired magnetic resonance data is therefore possible.

In a multislice acquisition, it may be sufficient in this case to acquire the navigator dataset for a single slice provided a prior navigator dataset is available as reference to which the navigator may be aligned. The navigator dataset may, however, also be acquired for all the slices in order to allow a particularly accurate three-dimensional coverage.

In addition or alternatively, a reference dataset may also be acquired for a parallel imaging procedure and/or for an undersampling as a control measure that may also be assigned to the same measure condition. The procedure is analogous to that described above with regard to FIG. 14. With parallel imaging, it makes sense in the case of greater movements to obtain new calibration parameters for calibrating the kernels or for calculating the coil sensitivities from the calibration dataset in order to prevent a faulty reconstruction of the magnetic resonance data acquired following the movement.

Other control measures may be assigned to measure conditions. Thus, for example, when using an acquisition protocol with at least one preparation module (e.g., for MT, FLAIR or ASL), it may be provided that the control measure relates to a repeated output of the preparation module. Here, too, it may be beneficial or even necessary following strong movement to play out RF preparation pulses again in order to avoid acquisition errors in subsequent magnetic resonance data.

For example, in the case of very strong and/or long-lasting movement, the control measure may also relate to a halting of the acquisition protocol (e.g., of the acquisition procedure). The acquisition in accordance with the acquisition protocol is then paused and may be continued again (e.g., in the present case, automatically) following termination of the movement. As already described above for the commencement of the acquisition using the acquisition protocol, it is provided that at least one measurement value is acquired for at least one radiofrequency pulse without a following acquisition time interval in a continuation interval (e.g., for restoring the steady state), the acquisition of magnetic resonance data being continued if the motion information indicates no further current movement. A good example in this case is a fit of coughing that may lead to a great deal of unusable magnetic resonance data in this time, but with the described approach, it is possible to wait for this to cease.

Given such a continuation, it is provided in the present case that initially, with the continuation, as described above, a navigator dataset and/or a calibration dataset are/is acquired in order to enable a possible repositioning of the patient to be taken into account.

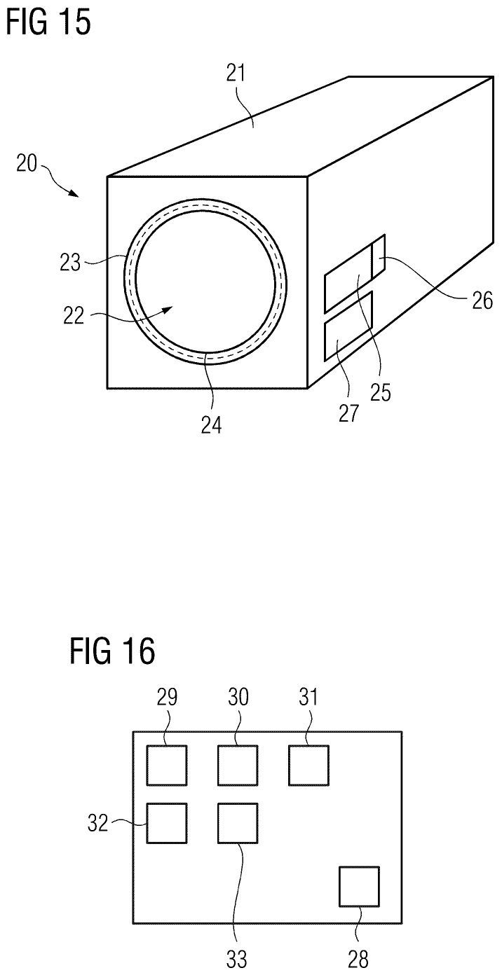

FIG. 15 shows a schematic diagram of a magnetic resonance device 20 according to the present embodiments. The magnetic resonance device 20 includes a main magnet unit 21 that in this case contains a superconducting main magnet (not shown in further detail) and a cylindrical patient receiving zone 22 into which a patient may be introduced by a patient couch (not shown in further detail here) for an acquisition procedure. In the present case, a gradient coil array 23 and a radiofrequency coil array 24 are provided surrounding the patient receiving zone 22. The radiofrequency coil array 24 may also be provided at least partly as a local coil arrangement. The radiofrequency coil array 24 is driven by a transmission chain 25 in order to output radiofrequency pulses 2, 4. Likewise, merely indicated in FIG. 15 is the measuring device 26 for acquiring measurement values that describe the back-reflected reflected power for at least one coil element of the radiofrequency coil array 24.

The operation of the magnetic resonance device 20 is controlled by a control device 27, the functional layout of which is described in more detail by FIG. 16. Accordingly, the control device 27 first includes a storage device 28, in which different data (e.g., also magnetic resonance data), cleaned time series 12, 13, 14, 15, motion information, and the like may be stored.

The control device 27 further includes a sequence unit 29 that controls the acquisition operation of the magnetic resonance device 20 and, in the present case, is also configured to actuate the measuring device 26 for the purpose of acquiring measurement values at least for correspondingly marked radiofrequency pulses of an acquisition protocol according to step S1. The preprocessing step S2 (e.g., including the corresponding substeps S2a to S2c and/or S2d and S2e) is performed in a preprocessing unit 30. As already mentioned, the preprocessing unit 30 may also include hardware components (e.g., for immediate pulse-specific correction, steps S2a to S2c).

According to step S3, the motion information may be determined in an evaluation unit 31. A reconstruction unit 32 serves for reconstructing image datasets from acquired magnetic resonance data and may be configured in accordance with the options described for step S4. A control unit 33 for other components of the magnetic resonance device 20 may also be configured for at least partly performing step S4. Finally, the sequence unit 29 may also be configured for at least partly performing step S4 (e.g., with regard to interventions in the workflow of the acquisition protocol or generally in the controlling of the acquisition procedure, such as for the reacquisition of motion-corrupted magnetic resonance data).

Independent of the grammatical term usage, individuals with male, female, or other gender identities are included within the term.

The elements and features recited in the appended claims may be combined in different ways to produce new claims that likewise fall within the scope of the present embodiments. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for operating a magnetic resonance device during an acquisition procedure in accordance with an acquisition protocol that uses at least one magnetic resonance sequence in an acquisition period and comprises radiofrequency pulses that precede acquisition time intervals, the method being computer-implemented and comprising:
acquiring at least one measurement value that describes a reflected power reflected back by a coil element that is used to output the radiofrequency pulses for at least some of the radiofrequency pulses in each case in a measurement interval, such that a time series of measurement values is produced over the acquisition period;
evaluating the time series of measurement values in an evaluation step, such that an occurring movement of the scanned examination subject is detected, the movement being described by motion information; and
using the motion information for controlling the acquisition procedure, in a reconstruction of an image dataset from acquired magnetic resonance data, or for controlling the acquisition procedure and in the reconstruction of the image dataset from the acquired magnetic resonance data,
wherein the time series is used in a preprocessing step prior to the evaluation step for at least partially cleaning the time series of a property using at least one piece of background information that describes an expected, non-motion-related property of the time series.

2. The method of claim 1, wherein the at least one measurement value is acquired at least for each radiofrequency pulse preceding an acquisition time interval.

3. The method of claim 1, wherein all the radiofrequency pulses for which a measurement value is acquired have at least one same characteristic variable, or the background information describes a characteristic variable differing between different radiofrequency pulses for which a measurement value is acquired.

4. The method of claim 3, wherein the characteristic variable relates to a flip angle described by the radiofrequency pulse, a pulse amplitude, a pulse phase, a pulse frequency, a pulse shape, or any combination thereof, the characteristic variable is determined from control information for generating the radiofrequency pulse, or a combination thereof.

5. The method of claim 3, wherein the measurement values in the preprocessing step relating to at least one measurement variable are normalized, characteristics information that assigns correction values to characteristic variables is used, the correction values describing occurring deviations caused by different values of the at least one characteristic variable due to the characteristics of the transmission chain used to output the radiofrequency pulses, or a combination thereof,
wherein the correction value is used for correcting the measurement values with respect to the deviations.

6. The method of claim 1, wherein initially, in the preprocessing step, the time series is subdivided based on at least one piece of time structure information of the at least one piece of background information into partial time series having a property expected for the respective partial time series, whereafter the cleaning is performed in a process specific to each partial time series.

7. The method of claim 6, wherein the at least one piece of time structure information comprises:
a subdivision of the acquisition protocol into repetitions;
a subdivision of the acquisition protocol into echo trains;
a subdivision of the acquisition protocol into sections containing slice groups having slices that are acquired immediately contiguously; and
a subdivision of the acquisition protocol into acquisition procedures for at least one slice.

8. The method of claim 6, wherein the time structure information comprises desired profile information, and
wherein the partial time series are corrected at least to some extent by a profile property described by the desired profile information.

9. The method of claim 8, further comprising performing at least one detrending in order to correct the profile property.

10. The method of claim 1, wherein in an acquisition in multiple slices, the motion information is determined on a slice-specific basis, individual k-space sections are identified by the motion information as affected by movement, or a combination thereof.

11. The method of claim 1, wherein the motion information is used during the reconstruction in order to exclude magnetic resonance data that was acquired in at least one time period affected by movement, for the reacquisition of said data, or a combination thereof.

12. The method of claim 11, wherein a reconstruction function compensating for missing magnetic resonance data is used for the reconstruction.

13. The method of claim 1, wherein the motion information is determined during the acquisition using the acquisition protocol for the purpose of controlling the acquisition procedure and is evaluated using at least one measure condition that is met when strength threshold values are exceeded by a strength of the current movement described by the motion information, and
wherein each measure condition is assigned a control measure that is actioned when the measure condition is met.

14. The method of claim 13, wherein for one or more of the at least one measure condition, the control measure relates to a fitting of the acquisition protocol to a next possible acquisition of a navigator dataset, a reference dataset for a parallel imaging procedure, an undersampling, or any combination.

15. The method of claim 14, wherein when a steady state is held by the output of radiofrequency pulses with following acquisition time intervals according to a timing sequence scheme, the timing sequence scheme is also maintained for the acquisition of the navigator dataset, the reference dataset, or the navigator dataset and the reference dataset.

16. The method of claim 13, wherein when at least one preparation module having at least one preparation pulse is used, the control measure of one of the at least one measure conditions relates to a repeated output of the preparation module.

17. The method of claim 13, wherein for one or more of the at least one measure condition, the control measure relates to a halting of the acquisition protocol.

18. A magnetic resonance device comprising:
a main magnet unit comprising a main magnet for generating a main magnetic field;
a gradient coil array; and a radiofrequency coil array to which there are assigned:
a transmission chain configured to output radiofrequency pulses during an acquisition procedure in accordance with an acquisition protocol that uses at least one magnetic resonance sequence in an acquisition period and comprises radiofrequency pulses that precede acquisition time intervals; and
a measuring device configured to measure a reflected power reflected back by a coil element of the radiofrequency coil array that is used to output the radiofrequency pulses,
wherein the magnetic resonance device further comprises a control device that comprises:
a sequence unit configured to:
control the acquisition procedure in accordance with the acquisition protocol; and
actuate the measuring device for the acquisition of at least one measurement value of the back-reflected reflected power for at least some of the radiofrequency pulses, in each case in a measurement interval, such that a time series of measurement values is produced over the acquisition period;
an evaluation unit configured to evaluate the time series of measurement values in an evaluation step for detection of an occurring movement of the scanned examination subject, the movement being described by motion information, wherein the sequence unit is further configured to use the motion information for control of the acquisition procedure, a reconstruction unit that is configured for use of the motion information in a reconstruction of an image dataset from acquired magnetic resonance data is provided, or a combination thereof; and
a preprocessing unit configured to clean the time series of the property at least to some extent in a preprocessing step prior to the evaluation step using at least one piece of background information that describes a non-motion-related, expected property of the time series.

19. In a non-transitory computer-readable storage medium that stores instructions executable by one or more processors to operate a magnetic resonance device during an acquisition procedure in accordance with an acquisition protocol that uses at least one magnetic resonance sequence in an acquisition period and comprises radiofrequency pulses that precede acquisition time intervals, the instructions comprising:
acquiring at least one measurement value that describes a reflected power reflected back by a coil element that is used to output the radiofrequency pulses for at least some of the radiofrequency pulses in each case in a measurement interval, such that a time series of measurement values is produced over the acquisition period;
evaluating the time series of measurement values in an evaluation step, such that an occurring movement of the scanned examination subject is detected, the movement being described by motion information; and
using the motion information for controlling the acquisition procedure, in a reconstruction of an image dataset from acquired magnetic resonance data, or for controlling the acquisition procedure and in the reconstruction of the image dataset from the acquired magnetic resonance data,
wherein the time series is used in a preprocessing step prior to the evaluation step for at least partially cleaning the time series of a property using at least one piece of background information that describes an expected, non-motion-related property of the time series.
